(19) United States
(12) Patent Application Publication
(10) Pub. No.: US 2009/0227487 A1
(43) Pub. Date: Sep. 10, 2009

(54) METHODS FOR THE CONTROL OF MACROPHAGE-ASSOCIATED INFLAMMATION

(76) Inventors: Jeremy Pearl, Stanford, CA (US); Ting Ma, Stanford, CA (US); William H. Robinson, Palo Alto, CA (US); R. Lane Smith, Stanford, CA (US); Stuart B. Goodman, Stanford, CA (US)

Correspondence Address: Stanford University Office of Technology Licensing;Bozicevic, Field & Francis LLP, 1900 University Avenue, Suite 200, East Palo Alto, CA, 94303, US

(21) Appl. No.: 12/397,925

(22) Filed: Mar. 4, 2009

Related U.S. Application Data
(60) Provisional application No. 61/034,082, filed on Mar. 5, 2008.

(51) Int. Cl. A61K 38/02; A61P 29/00

(52) U.S. Cl. 514/2

(57) ABSTRACT

Particle induced inflammatory diseases are treated by administration of an effective dose of an inhibitor of MyD88 adaptor protein.

### BACKGROUND OF THE INVENTION

Arthroplasty (literally “formation of joint”) is an operative procedure of orthopedic surgery, in which the arthritic or dysfunctional joint surface is replaced with something better or by remodeling or realigning the joint by osteotomy or some other procedure. For the last 45 years the most successful and common form of arthroplasty is the surgical replacement of arthritic or destructive or necrotic joint or joint surface with prosthesis. For example a hip joint that is affected by osteoarthritis may be replaced entirely with a prosthetic hip. These procedures can relieve pain, restore range of motion and improve walking ability. Indications for arthroplasty include arthritis; avascular necrosis or osteonecrosis; congenital dislocation of the hip joint; acetabular dysplasia (shallow hip socket); frozen shoulder, loose shoulder; traumatized and malaligned joint; and joint stiffness.

Total joint replacement is an effective treatment for relieving pain and restoring function for patients with damaged joints. Approximately 500,000 total hip and knee replacements are performed each year in the United States. These numbers will increase as the population continues to age and as the indications for joint arthroplasty extend to younger patients. For the majority of patients so treated, initial results following surgery are excellent.

Despite this success, implant wear remains the major problem facing the long-term success and survival of these artificial joints. Recent studies have shown that large amounts of minute wear particles are produced by these orthopaedic implants (both metal and plastic), setting into motion a cascade of events that ultimately may result in the disappearance of bone around the implant (osteolysis). This can lead to implant loosening and failure of the artificial joint. Surgery to replace these failures is more difficult to perform, is more costly, and has a poorer outcome than the original joint replacement surgery.

Aseptic loosening can occur over time as the result of inadequate initial fixation, mechanical loss of fixation over time, or biologic loss of fixation caused by particle-induced osteolysis around the implant. The causes of particle accumulation vary from implant interface wear, micromotion occurring in response to corrosion, oxidative reactions, and minor pathogen contaminations. In general, the initial response is a localized anti-inflammatory response that is characterized by formation of fibrous tissue that encapsulates the implant. Often, synovial fluid and synovial lining membranes are also formed, and granulomatous tissue is established. Immunohistochemical studies of these tissues have revealed an abundance of macrophages, fibroblasts, giant cells, neutrophils, and lymphocytes. However, aseptic loosening is characterized by poorly vascularized connective tissue dominated by fibroblasts and macrophages. Subsequently, secretion of proinflammatory factors, gelatinases, and proteases contributes to periprosthetic osteolysis and to failure of the joint implant.

Wear debris is formed at prosthetic joint articulations, modular interfaces, and nonarticulating interfaces. Although a wide range of particles has been found, the majority of particles formed are less than 5 μm in diameter and are randomly shaped. Clearly, particulate debris load and particle composition are important factors in the osteolytic process. Ongoing investigations have included evaluation of materials that are optimal in terms of minimizing particle generation over time, such as ceramics, and use of highly cross-linked polyethylene and of metal-on-metal articulations.

The major limiting factor for total joint replacement is the induction of osteolysis in the periprosthetic tissues by orthopedic biomaterial wear debris. The present invention addresses this problem by approaching the inflammatory response. The methods are of great interest for clinical use. The methods of the invention also find use in the selective control of inflammation induced by sepsis without substantial impairment of immune system function.

### SUMMARY OF THE INVENTION

The invention provides methods for treating particle induced inflammatory diseases involving macrophage activation, which may include inflammation associated with orthopedic biomaterial wear debris; sepsis; and the like. Such inflammation can lead to osteolysis in periprosthetic tissues and other undesirable sequelae. The methods of the invention use specific inhibitors to interact with and inactivate the MyD88 adaptor protein, which is activated by plasma membrane receptor interactions with particulate debris and/or bacterial products or components of cellular breakdown.

### BRIEF DESCRIPTION OF THE DRAWINGS

FIG. 1: TNF-alpha release after 1, 4, and 12 hours of macrophage incubation with media alone, PMMA particles, particles plus MyD88 inhibitor, and particles plus control peptide. * p<0.01 vs. media and inhibitor groups. ** p<0.01 vs. media and particle groups.

FIG. 2. TNF-alpha release after 1, 4, and 12 hours of incubation of wild type macrophages with media alone, wild type (WT) macrophages with PMMA particles, and MyD88−/− (KO) macrophages with PMMA particles. * p<0.01 vs. media and knockout groups. ** p<0.01 vs. media and wild type groups.

FIG. 3. TNFα production in WT, MyD88−/− and TRIF −/−/ macrophages.

FIG. 4. TNFα mRNA expression.

FIG. 5. TLR-4 mRNA expression.

FIG. 6. FIG. 1. TNF-alpha release after 12 hours of macrophage incubation with media alone, polymyxin B, LPS, and LPS plus polymyxin B. * p<0.01 vs. all other groups. ** p<0.01 vs. all other groups.

FIG. 7. TNF-alpha release after 1, 4, and 12 hours of macrophage incubation with media alone, PMMA particles alone, and PMMA particles with polymyxin B. * p<0.01 vs. media alone.

### DETAILED DESCRIPTION OF THE EMBODIMENTS

Before the present methods are described, it is to be understood that this invention is not limited to particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges, subject to any specifically excluded limit in the stated range. As used herein and in the appended claims, the singular forms “a”, “and”, and “the” include plural referents unless the context clearly dictates otherwise.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

### DEFINITIONS

The mononuclear phagocyte system is comprised of both circulating and fixed populations of cells. The circulating component is the monocyte. Upon migration into tissues these are referred to as histiocytes or tissue macrophages. The major fixed macrophages include: Sinusoidal lining cells of the spleen, lymph nodes, liver, and bone marrow; connective tissue histiocytes; mobile macrophages on serosal surfaces; alveolar macrophages within the lung; microglia in the nervous system; and mesangial macrophages within renal glomeruli. Macrophages produce a variety of substances that are involved in inflammation. Stimulation of macrophages can lead to induction of NO production, expression of iNOS and COX-2 protein, as well as up-regulating IL-6 release, and activating the transcription factor NF-κB.

The term “inflammatory” response as used herein refers to the development of a cellular response mediated by macrophages, etc. or their secretion products. In contrast to antigen specific responses, macrophages are activated by particulate debris and/or bacterial products or components of cellular breakdown.

“Specifically inhibit” the expression or activity of a protein shall mean to inhibit that protein's activity in a cell (a) more than the expression of any other protein. The biological activity of the targeted protein will usually be inhibited by at least about 10%, at least about 25%, at least about 75%, at least about 90%, at least about 95%, or more.

“Inhibiting” the expression of a gene in a cell shall mean either lessening the degree to which the gene is expressed, or preventing such expression entirely.

MyD88 (myeloid differentiation primary response gene 88); MYD88 is a dimeric protein, and contains a complete ‘death domain’ similar to the intracellular segment of TNF receptor-1. Human MYD88 cDNA encodes a 296-amino acid polypeptide with a predicted mass of 33 kD, sharing 81% amino acid identity with murine MyD88. The 150-amino acid C-terminal region has significant homology to the type I interleukin-1 receptor cytoplasmic domain. Northern blot analysis revealed that human MYD88 is expressed as 2 MYD88 hybridizing 1.6- and 3-kb mRNAs in a variety of tissues and cell lines. The genetic sequence of the human protein may be accessed at Genbank, NM_{—}002468, or as described by Hardiman et al. (1996) Oncogene 13 (11), 2467-2475.

Signaling by the human TOLL receptor employs MyD88 as an adaptor protein, and induces activation of NFKB via the IRAK kinase and the TRAF6 protein. The Toll-mediated signaling cascade using the NFKB pathway induces various immune response genes via this pathway. These findings have implicated MyD88 as a general adaptor/regulator molecule for the Toll/IL1R family of receptors for innate immunity.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom. The subject methods are used for prophylactic or therapeutic purposes.

The terms “individual,” “subject,” “host,” and “patient,” used interchangeably herein and refer to any mammalian subject for whom diagnosis, treatment, or therapy is desired, particularly humans. “Subject” or “patient” shall mean any animal, such as a human, non-human primate, mouse, rat, guinea pig or rabbit.

“Inhibiting” the onset of a disorder shall mean either lessening the likelihood of the disorder's onset, or preventing the onset of the disorder entirely. In the preferred embodiment, inhibiting the onset of a disorder means preventing its onset entirely. The methods of the invention are specifically applied to patients having a condition, e.g. a biomechanical implant, which predisposes to development of an inflammatory condition and osteolysis. Treatment is aimed at the treatment or prevention of osteolysis.

The term “biological sample” encompasses a variety of sample types obtained from an organism and can be used in a diagnostic or monitoring assay. The term encompasses blood, cerebral spinal fluid, and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

A “host cell”, as used herein, refers to a microorganism or a eukaryotic cell or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

“Comparable cell” shall mean a cell whose type is identical to that of another cell to which it is compared. Examples of comparable cells are cells from the same cell line.

“Diagnosis” as used herein generally includes determination of a subject's susceptibility to a disease or disorder, determination as to whether a subject is presently affected by a disease or disorder, prognosis of a subject affected by a disease or disorder, and use of therametrics (e.g., monitoring a subject's condition to provide information as to the effect or efficacy of therapy).

“Suitable conditions” shall have a meaning dependent on the context in which this term is used. That is, when used in connection with an antibody, the term shall mean conditions that permit an antibody to bind to its corresponding antigen. When this term is used in connection with nucleic acid hybridization, the term shall mean conditions that permit a nucleic acid of at least 15 nucleotides in length to hybridize to a nucleic acid having a sequence complementary thereto. When used in connection with contacting an agent to a cell, this term shall mean conditions that permit an agent capable of doing so to enter a cell and perform its intended function. In one embodiment, the term “suitable conditions” as used herein means physiological conditions.

Orthopaedic biomaterial wear debris. Orthopaedic biomaterials can be implanted into or near bones to facilitate healing or to compensate for a lack or loss of bone tissue. The materials used in orthopaedic surgery include, for example, ceramics; polymers; metals, such as stainless steel, cobalt-chromium and titanium; and restorable materials, such as biogas, various modifications of hydroxyapatite and bone grafts. Polyethylene and polymethylmethacrylate are commonly used in joints such as knee, elbow and hip replacements.

Following implantation of an orthopedic device, debris particles can be generated, usually from the plastic component, e.g. ultra high molecular weight polyethylene; UHMWPE; etc. from mechanical wear of the prosthesis. Studies have suggested that the cellular response to particles may vary with size, shape, composition, charge, and number of particles. Small polymethylmethacrylate (PMMA) and polyethylene particles (<20 μm) elicite an inflammatory cytokine response by macrophages, as indicated by increased release of tumor necrosis factor (TNF), IL-1, IL-6, prostaglandin (PG)E₂, matrix metalloproteinases, and other factors. Direct interactions between particle and cell surface are sufficient to activate osteoclastogenic signaling pathways. The rate at which particles accumulate is also considered an important factor in the occurrence of osteolysis.

Systemic inflammatory response syndrome”, or “SIRS”, refers to a clinical response to a variety of severe clinical insults, for example as manifested by two or more of the following conditions within a 24-hour period: body temperature greater than 38° C. or less than 36° C.; heart rate (HR) greater than 90 beats/minute; respiratory rate (RR) greater than 20 breaths/minute, or P_{CO2} less than 32 mm Hg, or requiring mechanical ventilation; and white blood cell count (WBC) either greater than 12×10⁹/L or less than 4×10⁹/L or having greater than 10% immature forms (bands). SIRS may result from a variety of conditions, including trauma such as burns or other insults, including sepsis.

Sepsis refers to a serious infection, localized, bacteremic or fungal, that is accompanied by systemic manifestations of inflammation. The term “onset of sepsis” refers to an early stage of sepsis, i.e. prior to a stage when the clinical manifestations are sufficient to support a clinical suspicion of sepsis. “Severe sepsis” refers to sepsis associated with organ dysfunction, hypoperfusion abnormalities, or sepsis-induced hypotension. Hypoperfusion abnormalities include, but are not limited to, lactic acidosis, oliguria, or an acute alteration in mental status. “Septic shock” refers to sepsis-induced hypotension that is not responsive to adequate intravenous fluid challenge and with manifestations of peripheral hypoperfusion.

Bacteremia and sepsis are closely related conditions. Bacteremia denotes bacteria in the bloodstream. Sepsis refers to a serious infection, localized, bacteremic or due to fungal infections, that is accompanied by systemic manifestations of inflammation. Septic shock is sepsis with hypoperfusion and hypotension refractory to fluid therapy. The more general term, systemic inflammatory response syndrome, recognizes that several severe conditions, including infections, pancreatitis, burns, trauma, etc. can trigger an acute inflammatory reaction, the systemic manifestations of which are associated with release into the bloodstream of a large number of endogenous mediators of inflammation.

Transient bacteremia may be caused by surgical manipulation of infected oral tissues or even routine dental manipulations; catheterization of an infected lower urinary tract; incision and drainage of an abscess; and colonization of indwelling devices, especially IV and intracardiac catheters, urethral catheters, and ostomy devices and tubes; and the like. Gram-negative bacteremia is typically intermittent and opportunistic; although it may have no effect on a healthy person, it can be seriously important in immunocompromised patients with debilitating underlying diseases, after chemotherapy, and in settings of malnutrition.

When bacteremia or infections with certain fungi produce changes in circulation such that tissue perfusion is critically reduced, septic shock ensues. Septic shock is most common with infections by gram-negative organisms, staphylococci, or meningococci. Septic shock is characterized by acute circulatory failure, usually with or followed by hypotension, and multiorgan failure.

Unless otherwise apparent from the context, all elements, steps or features of the invention can be used in any combination with other elements, steps or features.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### METHODS OF THE INVENTION

The invention provides methods for treating particle induced inflammatory diseases involving macrophage activation, which may include inflammation associated with orthopedic biomaterial wear debris; sepsis; and the like. Such inflammation can lead to osteolysis in periprosthetic tissues and other undesirable sequelae. The methods of the invention use specific inhibitors to interact with and inactivate the MyD88 adaptor protein, which is activated by plasma membrane receptor interactions with particulate debris and/or bacterial products or components of cellular breakdown.

In this invention, administering an effective dose of a MyD88 inhibitor can be effected or performed using any of the various methods and delivery systems known to those skilled in the art. The administering can be performed, for example, intravenously, orally, via implant, transmucosally, transdermally, intramuscularly, intrathecally, and subcutaneously. The delivery systems employ a number of routinely used pharmaceutical carriers.

In some embodiments of the invention, a subject is treated following implantation of a prosthetic device susceptible to the generation of wear debris, where the treatment may be initiated at any time, e.g. following detection of wear debris, detection of inflammatory reactions, and the like. Biologic and mechanical factors have been incriminated in the early and late stages of the development of osteolysis following joint replacement. Inflammatory reactions develop at early stages as part of the resolution process. These reactions primarily include increased circulation and elevated fluid levels in the vicinity of the affected tissue.

The cellular response is dominated by phagocytes and macrophages, although the osteolytic response includes various cell types, such as osteoclasts, fibroblasts, and osteoblasts/stromal cells. It is believed that recognition of particles relies on phagocytosis of small-sized particles by macrophages and unidentified cell surface interactions. The latter interactions may include nonspecific physical induction of transmembrane proteins or recognition of cell surface molecules by particles or proteins/factors that are adherent to the surface. Host cells recognize particles and release large quantities of proinflammatory cytokines and factors, including TNF, IL-1α and IL-1β, IL-6, RANKL and PGE₂, among others.

In other embodiments, an effective dose of an inhibitor of MyD88 is administered to a subject suffering from, or at risk of developing, bacteremia or sepsis. In some embodiments a patient is diagnosed with bacterimia or sepsis prior to administration of the MyD88 inhibitor.

### Therapeutic Agents

MyD88 is an adaptor protein, which plays an essential role in the intracellular signaling elicited by IL-1R and several TLRs. Central to its function is the ability of its Toll/IL-1R translation initiation region (TIR) domain to heterodimerize with the receptor and to homodimerize with another MyD88 molecule to favor the recruitment of downstream signaling molecules such as the serine/threonine kinases IL-1R-associated kinase 1 (IRAK1) and IRAK4.

The present invention contemplates a variety of MyD88 inhibitors that are employed to inhibit the biological activity of MyD88. These inhibitors may be administered to a mammal by any suitable means, such as those set forth in the various ensuing embodiments. Such inhibitors may include any compound, pharmaceutical, or other composition that effects an inhibition of the biological activity of MyD88. Such a composition may be administered to a mammal in an effective amount and by any suitable means, including, but not limited to, orally, topically, intravenously, intramuscularly, via a surgical device, such as a catheter, or via an implantable mechanism, such as a stent.

Peptide inhibitors of MyD88 dimerization are known in the art and commercially available, including the peptide DRQIKIWFQNRRMKWKKRDVLPGT; a synthetic peptido-mimetic compound (ST2825) modeled after the structure of a heptapeptide in the BB-loop of the MyD88-TIR domain, which interferes with MyD88 signaling (see Loiarro et al. (2007) J. Leukoc. Biol., Vol. 82, Issue 4, 811-812, herein specifically incorporated by reference); peptide inhibitors and peptidomimetics described in WO/2006/067091, herein specifically incorporated by reference; U.S. Patent application US20030148986, herein specifically incorporated by reference, describes various ways of inhibiting the expression or the biological activity of the protein MyD88, including the use of peptidomimetic agents that prevent the signalling of the protein. This inhibition is accomplished with small peptides (10-20 amino acids) that bind to the TLR-4 receptors, thus preventing the binding with MyD88. Small overlapping segments (approximately 10-20 amino acids) of MyD88 can be separated to test to see which of these prevent the transduction of the MyD88 cell signal by binding to the TLR-4 receptors. After the separation, the segments are duplicated and tested to determine whether the segment comprises at least one portion of MyD88 that binds to the TLR-4 receptor, which will prevent the binding of MyD88 and the transduction of the cell signal. Peptide inhibitors include ST2348 MyD88 (AC-EDVLPGT-NH₂); and ST2350 IL-18E (Ac-EDWPGG-NH₂). Peptide ST2348 has been conjugated with a fragment of the An-tennapaedia (Ap) protein with the sequence RQIKIWFQNRRMKWKK.

As an alternative to peptide inhibitors, appropriate viral vectors that can express antisense MyD88 RNA include expression vectors based on recombinant adenoviruses, adeno-associated viruses, retroviruses or lentiviruses, though non-viral vectors may be used, as well. Alternatively, a ribozyme-viral (adeno, adeno-associated or lentiviral) or non-viral vector against MyD88 mRNA in a mammal may be administered. Ribozymes are sequence-specific endoribonucleases that catalytically cleave specific RNA sequences, resulting in irreversible inactivation of the target mRNA, thereby inhibiting the gene expression. Ribozymes offer advantages over antisense ODN. For instance, ribozymes possess higher catalytic activity than ODN; a comparatively smaller quantity of ribozyme-containing active is thus required for inhibition of gene expression. Ribozymes can be delivered exogenously or can be expressed endogenously with the use of appropriate promoters in a viral vector. Methods of the present invention utilize a hammerhead ribozyme directed to human MyD88 mRNA. Desired quantity or the length of expression of the ribozyme-viral or non-viral vector can readily be determined by routine experimentation, as can the most effective and/or convenient route of administration.

Anti-sense oligonucleotides (ODN) or RNAi may be used to inhibit expression of MyD88. ODN can be synthesized on a nucleic acid synthesizer, such as the EXPIDITE Nucleic Acid Synthesizer (available from Applied Biosystems, Inc., Rockville, Md.) and purified using standard protocols. RNAi use dsRNA that are sufficiently homologous to a portion of the MyD88 gene product such that the dsRNA degrades mRNA that would otherwise affect the production of MyD88. siRNA, a well-defined 21-base duplex RNA may operate in conjunction with various cellular components to silence the MyD88 gene product with sequence homology. Efficient gene silencing may be achieved by employing siRNA duplexes which include sense and antisense strands each including approximately 21 nucleotides, and further paired such that they possess about a 19-nucleotide duplex region and about a 2-nucleotide overhang at each 3′ terminus. It will be appreciated by one of skill in the art of RNAi that alternately sized sense or antisense strands and/or variations on the size of the duplex and the overhang region that comprise them may be suitable for use with the methods of the present invention, and are contemplated as being within the scope thereof. Such appropriate alternate sizes may be readily ascertained without undue experimentation by one possessing such skill. Furthermore, the inclusion of symmetric 3′-terminus overhangs may aid in the formation of specific endonuclease complexes (“siRNPs”) with roughly equivalent ratios of sense and antisense target RNA cleaving siRNPs. The siRNA duplexes used in accordance with the present invention may be introduced to a cell via an appropriate viral or non-viral vector.

In another aspect, and antibody specific for MyD88 is utilized in treatment. Any suitable anti-MyD88 antibody may be used in conjunction with this aspect of the present invention, including, but in no way limited to, anti-MyD88 antibodies, and any suitable derivatives thereof, equivalents thereof, or compounds with active sites that functions in a manner similar to anti-MyD88 antibodies, whether those compounds are naturally occurring or synthetic (all hereinafter included within the term “anti-MyD88 antibody”). An appropriate quantity of an anti-MyD88 antibody necessary to effect the method of the present invention, and the most convenient route of delivering the same to a mammal may be determined by one of ordinary skill in the art, without undue experimentation. Furthermore, it will be readily appreciated by one of such skill that an anti-MyD88 antibody may be formulated in a variety of pharmaceutical compositions, any one of which may be suitable for use in accordance with the method of the present invention. Such an antibody may be delivered to a mammal through any conventional mechanism in an amount effective to inhibit MyD88 signaling in a mammal; the mechanism of delivery and quantity of antibody necessary for inhibiting MyD88 expression both being readily ascertainable without undue experimentation.

The MyD88 inhibitor, which may be a polypeptide, or a functional fragment or variant thereof is administered to a patient. A “variant” polypeptide means a biologically active polypeptide as defined below having less than 100% sequence identity with a native sequence polypeptide. Such variants include polypeptides wherein one or more amino acid residues are added at the N- or C-terminus of, or within, the native sequence; from about one to forty amino acid residues are deleted, and optionally substituted by one or more amino acid residues; and derivatives of the above polypeptides, wherein an amino acid residue has been covalently modified so that the resulting product has a non-naturally occurring amino acid. Ordinarily, a biologically active variant will have an amino acid sequence having at least about 90% amino acid sequence identity with a native sequence polypeptide, preferably at least about 95%, more preferably at least about 99%. The sequence of MyD88 inhibitor peptides as described above may be altered in various ways known in the art to generate targeted changes in sequence. The sequence changes may be substitutions, insertions or deletions. Such alterations may be used to alter properties of the protein, by affecting the stability, specificity, etc. Techniques for in vitro mutagenesis of cloned genes are known. Examples of protocols for scanning mutations may be found in Gustin et al., Biotechniques 14:22 (1993); Barany, Gene 37:111-23 (1985); Colicelli et al., Mol Gen Genet. 199:537-9 (1985); and Prentki et al., Gene 29:303-13 (1984). Methods for site specific mutagenesis can be found in Sambrook et al., Molecular Cloning: A Laboratory Manual, CSH Press 1989, pp. 15.3-15.108; Weiner et al., Gene 126:35-41 (1993); Sayers et al., Biotechniques 13:592-6 (1992); Jones and Winistorfer, Biotechniques 12:528-30 (1992); Barton et al., Nucleic Acids Res 18:7349-55 (1990); Marotti and Tomich, Gene Anal Tech 6:67-70 (1989); and Zhu Anal Biochem 177:120-4 (1989).

Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

The MyD88 inhibitor peptides may be prepared by in vitro synthesis, using conventional methods as known in the art. Various commercial synthetic apparatuses are available, for example, automated synthesizers by Applied Biosystems, Inc., Foster City, Calif., Beckman, etc. By using synthesizers, naturally occurring amino acids may be substituted with unnatural amino acids. The particular sequence and the manner of preparation will be determined by convenience, economics, purity required, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

Active polypeptides or polynucleotides can serve as the active ingredient in pharmaceutical compositions formulated for the treatment of various disorders as described above. The active ingredient is present in a therapeutically effective amount, i.e., an amount sufficient when administered to substantially modulate the effect of the targeted protein or polypeptide to treat a disease or medical condition mediated thereby. The compositions can also include various other agents to enhance delivery and efficacy, e.g. to enhance delivery and stability of the active ingredients.

Thus, for example, the compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The MyD88 inhibitor peptides may be administered in a single dose, or in multiple doses, usually multiple doses over a period of time, e.g. daily, every-other day, weekly, semi-weekly, monthly etc. for a period of time sufficient to reduce severity of the disease, which may comprise 1, 2, 3, 4, 6, 10, or more doses.

Determining a therapeutically or prophylactically effective amount an agent that provides MyD88 inhibitor activity can be done based on animal data using routine computational methods. In one embodiment, the therapeutically or prophylactically effective amount contains between about 0.1 mg and about 1 g of protein. In another embodiment, the effective amount contains between about 1 mg and about 100 mg of protein, as applicable. The effective dose will depend at least in part on the route of administration. The agents may be administered orally, in an aerosol spray; by injection, e.g. i.m., s.c., i.p., i.v., etc. The dose may be from about 0.1 μg/kg patient weight; about 1 μg/kg; about 10 μg/kg; to about 100 μg/kg.

Treating, treatment, or therapy of a disease or disorder shall mean lessening the severity of adverse clinical symptoms by administration of a MyD88 inhibitor peptide composition. As used herein, ameliorating a disease and treating a disease are equivalent.

The method also provide for combination therapy, where the combination may provide for additive or synergistic benefits.

### Animal Models

In one embodiment, the present invention utilizes a non-human animal model for MyD88 function. In some embodiments the animal model comprises a homozygous deletion of MyD88 gene, which may be compared to a wild animal, an animal having a homozygous deletion of the TRIF gene, and the like. Such knockout animals are characterized by lacking a functional protein, which may be truncated, mutated, substantially absent, etc. Animals expressing a mutated form of a gene or overexpressing a natural form of a gene by either “knock-in” technology or transgenesis may also be used. A number of animal models of osteolysis, as are known in the art, can be developed with wild-type or knockout animals.

Mouse models of interest include the air pouch model, in which bone tissue is implanted and then undergoes resorption. Polyethylene particles are then introduced into the pouch to promote inflammation and osteolysis.

The murine calvarial model has served as an important in vivo surrogate to understand the biologic effects of particles and the mechanisms involved in inflammatory bone resorption. In this model, particles of, for example, titanium or polyethylene are implanted onto the calvaria, which leads to profound inflammation, osteoclast formation, and bone resorption. The resulting bone lass can be measured quantitatively, allowing assessment of the potential of various genetic approaches and biologic agents to prevent bone loss. The model permits use of transgenic and knockout approaches in which the role of specific genes can be assessed, offering significant advantages over other approaches, including the use of larger animal models. Other strengths include the rapidity of the development of osteolysis (about 10 days), the relatively low cost, and the ability to screen a large number of compounds and doses of various agents.

The tibial hemiarthroplasty model is also of interest, where animals are implanted with a tibial hemiarthroplasty, e.g. of an implant or particle beaded device (e.g. titanium, polyethylene, etc. Although radiographic findings associated with osteolysis are not routinely demonstrated with this model, a significant infiltration of macrophages occurs in the periprosthetic tissue, with an increase in osteoclasts and Howship lacunae and a decrease in new bone formation in the surrounding tissue when polymethylmethacrylate (PMMA) particles are introduced into the periprosthetic tissue at the time of implantation. Alternatively an osmotic pump can be used for continuous introduction of particles, e.g. in a femoral Implant With particles.

The animal models are useful for screening candidate therapeutic agents and treatment modalities. Through use of the subject animals or cells derived therefrom, one can identify ligands or substrates that affect the macrophage induced osteolysis.

Drug screening protocols may include a panel of animals, for example a test compound or combination of test compounds, and negative and/or positive controls, where the positive controls may be known MyD88 acting agents. Such panels may be treated in parallel, or the results of a screening assay may be compared to a reference database.

Depending on the particular assay, whole animals may be used, or cells derived therefrom. Cells may be freshly isolated from an animal, or may be immortalized in culture. Candidate therapies may be novel, or modifications of existing treatment options. For screening assays that use whole animals, a candidate agent, or treatment is applied to the subject animals. Typically, a group of animals is used as a negative, untreated or placebo-treated control, and a test group is treated with the candidate therapy. Generally a plurality of assays are run in parallel with different agent dose levels to obtain a differential response to the various dosages. The dosages and routes of administration are determined by the specific compound or treatment to be tested, and will depend on the specific formulation, stability of the candidate agent, response of the animal, etc.

The analysis may be directed towards determining effectiveness in prevention of osteolysis induction. Alternatively, the analysis is directed toward regression of existing conditions, and the treatment is administered after initial onset of osteolysis.

In either case, after a period of time sufficient for the development or regression of the disease, the animals are assessed for impact of the treatment, by visual, histological, immunohistological, and/or other assays suitable for determining effectiveness of the treatment. The results may be expressed on a semi-quantitative or quantitative scale in order to provide a basis for statistical analysis of the results.

The term “agent” as used herein describes any molecule, e.g. protein or pharmaceutical, with the capability of affecting MyD88 activity, particularly inhibiting MyD88 dimerization. An agent or treatment is administered to an animal of the invention, or to cells derived therefrom. Pharmaceutical agents, antibodies, other proteins, etc. are of interest. In some embodiments, a candidate agent is obtained and the animal models used to validate its effectiveness.

A candidate agent may be any molecule, e.g. protein or pharmaceutical, with the capability of altering MyD88 activity. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Candidate agents also encompass numerous classes of proteins, including antibodies in intact, truncated, or otherwise modified forms.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example. A number of different types of combinatorial libraries and methods for preparing such libraries have been described, including for example, PCT publications WO 93/06121, WO 95/12608, WO 95/35503, WO 94/08051 and WO 95/30642, each of which is incorporated herein by reference.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

For example. preliminary screens can be conducted by screening for compounds capable of interfering with the MyD88 dimerization, as at least some of the compounds so identified are likely inhibitors. The binding assays can involve contacting a combination of proteins with one or more test compounds and allowing sufficient time for the proteins to form a complex. Any complexes formed can be detected using any of a number of established analytical techniques. Protein binding assays include, but are not limited to, methods that measure co-precipitation, co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89.

Compounds that are initially identified by any of the foregoing screening methods can be further tested to validate the apparent activity. The basic format of such methods involves administering a lead compound identified during an initial screen to an animal that serves as a model for humans and then determining the initiation and/or progression of disease. The animal models utilized in validation studies generally are mammals. Specific examples of suitable animals include, but are not limited to, primates, mice, and rats.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

### EXPERIMENTAL

### Example 1

Aseptic loosening of joint replacements is the most common cause of revision surgery. The etiology is related to wear particles from the implant, which produce chronic inflammation resulting in periprosthetic osteolysis (Looney et al. Arthritis Res 4, 59-63, 2002). Macrophage activation is a key component of this inflammatory response. One pathway for macrophage activation involves the innate immune system via activation of Toll-Like Receptors (TLR). Many of the effects of TLR's are due to interaction with Myeloid Differentiation primary response gene 88 (MyD88), an adapter protein which couples the TLR to downstream signaling kinases, eventually culminating in activation of the transcription factor NFκB (Akira et al. Nature Rev Immunol 4, 499-511, 2004). Wear debris-induced inflammatory osteoclastogenesis requires cytokine production and osteoclast differentiation via the NFκB pathway (Lind et al. Cytokine 12, 909-913, 2000). The current study investigates whether MyD88 plays a role in the macrophage inflammatory response to wear-debris recognition.

### Materials and Methods

MyD88 inhibitory peptide experiments: The murine monocyte/macrophage cell line Raw 264.7 was cultured in DMEM containing 10% (v/v) FBS (5% CO₂, 37° C.). Cells were plated in 24-well tissue culture plates at 1×10⁵ cells/well in 1 ml of media with serum and allowed to adhere for 24 hours. Cells were then preincubated for 24 hours with 1 ml of fresh media containing one of the following treatments: 1) 100 μM MyD88 homodimerization inhibitory peptide (Imgenex), which binds the MyD88 monomer, blocking MyD88 activation; 2) 100 μM control peptide, which crosses the cell membrane but does not interact with MyD88; 3) no peptide. Following the preincubation period in each group, PMMA particles (Polysciences, 1-10 μm) were added at a dose of 0.30% v/v. Each group was plated in triplicate and the experiment was repeated three times.

Quantification of TNF-α release: Macrophages were exposed to PMMA particles and samples from the culture media were collected at 1, 4, and 12 hours post challenge. TNF-α levels were quantified using ELISA kits. Data were analyzed by ANOVA.

MyD88−/− cell experiments: Bone marrow derived macrophages isolated from the femurs of C57BL/6 wild type (WT) and MyD88^{-/-} knockout mice. Cells were cultured in 24-well plates at a density of 8×10⁵ cells/well in 1 ml of RPMI-1640 media for 24 hours to allow adherence. The media was then exchanged with 1 ml of fresh media containing PMMA particles at a dose of 0.30% v/v. TNF-α levels were quantified as above.

Preparation of particles: PMMA particles were washed 5× with 70% ethanol and incubated overnight with shaking at 4° C. The particles were then washed 3× with DPBS and resuspended to a concentration of 16% v/v. The particles were free of endotoxin using a high-sensitivity Limulus amoebocyte lysate assay (BioWhittaker).

### Results

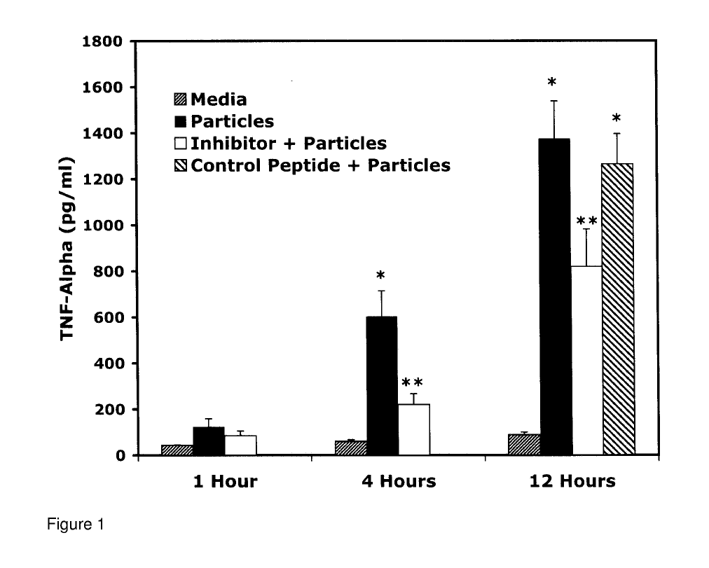

MyD88 inhibitory peptide decreases particle-induced TNF-α release: Macrophages incubated without particles had no significant TNF-α release. In the presence of PMMA particles, Raw264.7 macrophages exhibited a time-dependent increase in TNF-α release as early as 4 h post particle exposure (p<0.01) (FIG. 1). The MyD88 inhibitory peptide significantly decreased particle-induced TNF-α release, by 63% at 4 h (p<0.01) and 32% at 12 h (p<0.01). The control peptide had no significant effect compared to the particle only treated group (FIG. 1).

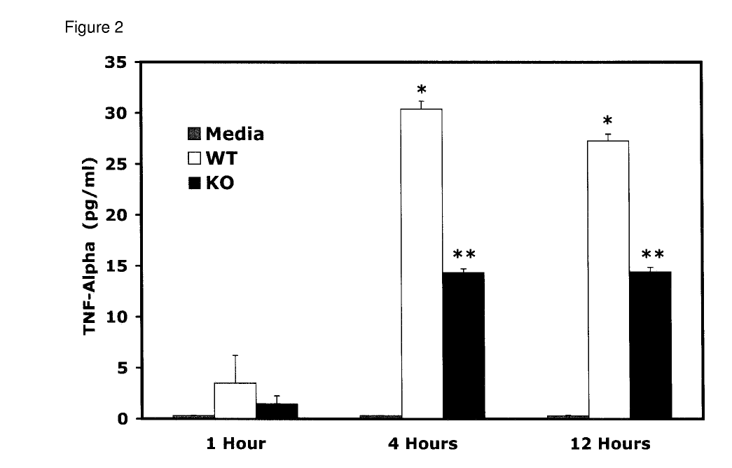

MyD88−/− macrophages have decreased particle-induced cytokine release: Bone marrow derived macrophages from wild-type mice had increased TNF-α release following exposure to PMMA particles for 4 hours (p<0.01) or 12 hours (p<0.01) (FIG. 2). MyD88^{-/-} macrophages exhibited significantly less TNF-α release at both time points, with a 53% decrease at 4 h (p<0.01) and a 47% decrease at 12 h (p<0.01) relative to wild type cells (FIG. 2).

### Discussion

This study demonstrates that in both the macrophage cell line Raw264.7 and in C57BL/6 bone marrow derived femoral macrophages, the response to PMMA particles is dependent upon the adapter molecule MyD88, as part of Toll-Like Receptor signaling. The particle induced increase in TNF-α production was decreased by approximately half when MyD88 signaling was disrupted by either an inhibitory peptide which blocks MyD88 activation or by disruption of the MyD88 gene. The roles of specific TLR's and of TLR signaling through MyD88-independent pathways such as TRIF can be examined. The TLR pathway of the innate immune system represents a novel therapeutic target for prevention and treatment of particle associated periprosthetic osteolysis.

### Example 2

### Role of TRIF and MyD88 in PMMA Particle Induced Pro-Inflammatory Signaling

When TLR-4 is stimulated it can activate NFκB by interacting with either MyD88 or TRIF. We first investigated whether disruption of the MyD88 gene and subsequent exposure to wear debris particles alters the in vitro expression of TLR-4 and TNF-α. Similarly, we exposed cells with either MyD88 or TRIF disrupted to wear-debris particles, comparing their in vitro production of the pro-inflammatory cytokine TNF-α.

Methods: PMMA particles (Polysciences) were washed with 70% ethanol and demonstrated free of endotoxin using a Limulus amoebocyte lysate assay (BioWhittaker).

KO cell experiments: Bone marrow derived macrophages isolated from C57BL/6 wild type (WT), MyD88−/− and TRIF−/− mice were cultured at a density of 8×10⁵ cells/well for 24 hours to allow adherence. The media was then replaced with fresh media containing PMMA particles at a dose of 0.30% v/v.

Quantification of TNF-α release: WT, MyD88−/−, TRIF−/− cells were exposed to PMMA particles, samples from the culture media were collected at 1, 4, and 12 h post challenge. TNF-α levels were quantified using ELISA kits. Each group contained an N=6.

RT-PCR analysis: Total RNA was extracted from particle challenged WT and MyD88−/− (referred to as KO in the figures) cells. RT-PCR was performed using probes for GAPDH, TNF-α and TLR-4. Relative quantification was measured with the delta comparative threshold method comparing reference (GAPDH) to target genes expression.

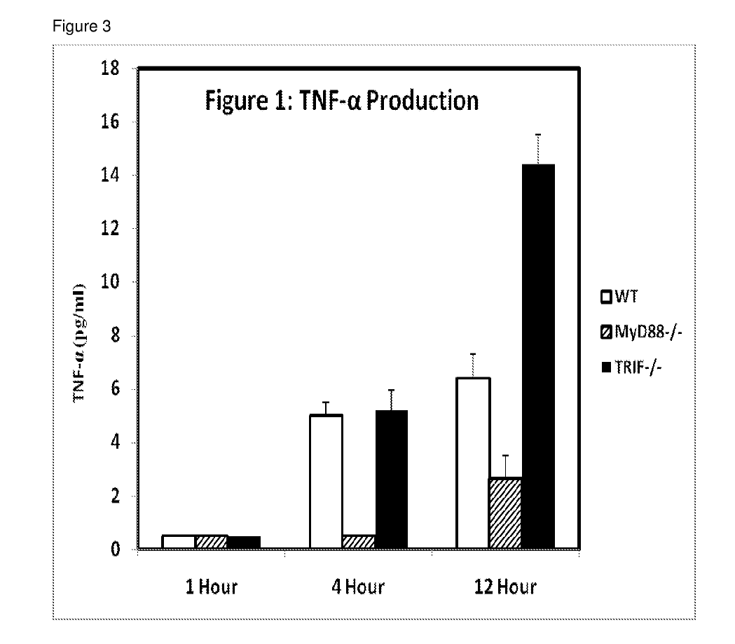

Results: TNF-α release from WT and KO cells: WT Macrophages exposed to PMMA particles exhibited a time-dependent increase in the release of TNF-α that was significant at 4 h post particle exposure and remained elevated at 12 h (FIG. 3). There was a marked decrease in TNF-α release in the MyD88−/− cells and increase in the TRIF−/− cells compared to WT cells (FIG. 3).

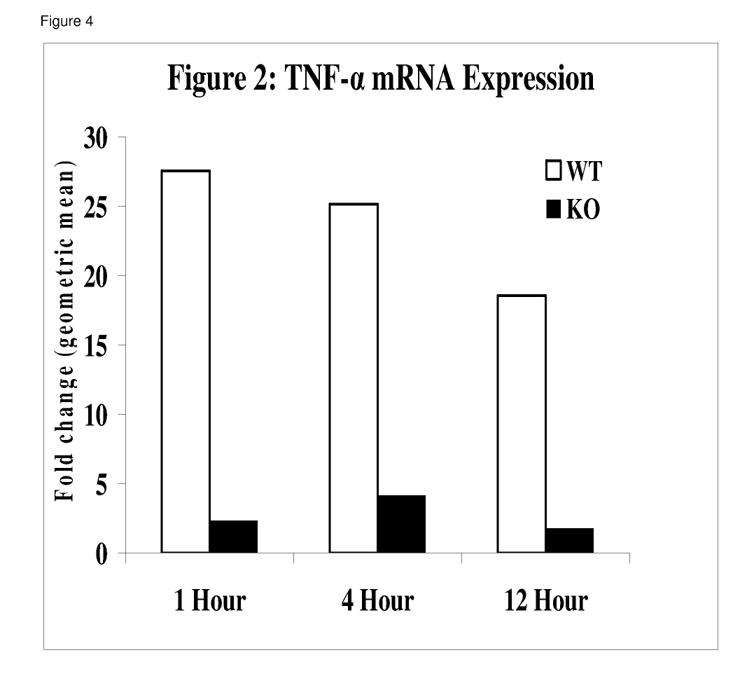

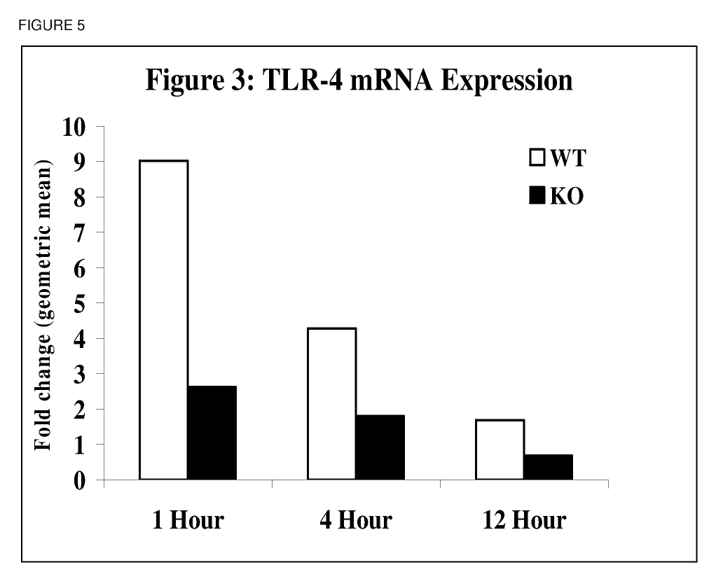

Comparison of Expression Profiles: Addition of PMMA particles to WT cells markedly increased TNF-α gene expression levels at all time points (FIG. 4). TNF-α expression was markedly higher in WT compared to KO cells at all time points. Addition of PMMA particles to WT cells increased TLR-4 expression levels at 1 h, but the effect became non-significant by 12 h. (FIG. 5). TLR-4 expression in MyD88−/− cells demonstrated a small increase over baseline at 1 h, and significantly decreased over time. The trend towards increased levels of TLR-4 expression in the WT group compared to the MyD88−/− (KO) group did not reach statistical significance. However, baseline expression levels of TLR-4 were significantly increased (40-fold) between the WT and MyD88−/− group.

Conclusions: This study demonstrates that the response to PMMA particles is partly dependent upon MyD88, presumably as part of TLR signaling. TNF-α production in response to PMMA particles was markedly diminished upon disruption of MyD88, in contrast disruption of TRIF increased TNF-α production. Presumably secondarily to a compensatory increase in MyD88 expression resulting in a more robust response upon PMMA particle induced stimulation of TLR-4. To investigate the involvement of TLR-4 in recognition of wear debris particles, we assayed changes in gene expression. TLR4 may be involved since disruption of the adapter molecule MyD88 impaired particle-induced TLR-4 upregulation. TLR signaling through MyD88 may be a novel therapeutic target for prevention of particle induced periprosthetic osteolysis.

### Example 3

### PMMA Particles can Activate Macrophages Independent of Adherent Endotoxin

Aseptic loosening is the major cause of long-term failure of orthopaedic implants for joint replacement, resulting in approximately 40,000 revision surgeries annually in the United States. Wear particles released from the implant are phagocytosed by macrophages and stimulate the release of bone resorptive cytokines, leading to loss of implant fixation (Maloney et al. J Bone Joint Surg 77A, 1448-61, 1995). The biological activity of debris particles may vary with their composition, size, morphology, concentration and surface adsorbed serum-derived proteins (Gonzalez et al. J Biomed Mater Res 30, 463-73, 1996). Previous studies have focused on the role that adsorbed endotoxin, the lipopolysaccharide (LPS) from the cell wall of gram-negative bacteria, may play in the biological activity of wear particles. Some investigators have claimed that treatments which remove adsorbed endotoxin markedly decrease or completely reverse the biological activity of the particles (Bi et al. J Bone Mineral Res 16, 2082-91, 2001; Daniels et al. J Biomed Mater Res 49, 469-78, 2000); others have argued that the impact of these treatments is due to alterations in the particles themselves rather than to removal of endotoxin. Polymyxin B is an antibiotic used for treatment of gram-negative septic shock and works by binding and neutralizing LPS. The current study used polymyxin B treatment to bind and neutralize LPS as a means for determining the presence and significance of adherent endotoxin on the effects of wear debris particles.

### Materials and Methods

Preparation of particles: PMMA particles were washed 5× with 70% ethanol and incubated overnight with shaking at 4° C. The particles were then washed 3× with DPBS and resuspended to a concentration of 16% v/v. The particles were free of endotoxin using a high-sensitivity Limulus amoebocyte lysate assay (BioWhittaker).

Polymyxin B preparation: Polymyxin B was prepared fresh before each use and added to cell cultures at a concentration of 10 μg/ml. This concentration has been shown to decrease LPS-induced inflammatory reactions without causing significant toxicity.

Raw264.7 macrophage cell culture experiments: The murine monocyte/macrophage cell line Raw 264.7 was cultured in DMEM containing 10% (v/v) FBS (5% CO₂, 37° C.). Cells were plated in 24-well tissue culture plates at 1×10⁵ cells/well in 1 ml of media with serum and allowed to adhere for 24 hours. The media was then replaced with 1 ml of media containing one of four conditions: 1) media alone; 2) 10 μg/ml of polymyxin B; 3) 500 pg/ml of LPS (E. coli 055:B5, Sigma); 4) 500 pg/ml LPS and 10 μg/ml polymyxin B. TNF-α release was measured at 12 h after incubation using ELISA. Each group was plated in triplicate and the experiment was repeated two times. Data were analyzed by ANOVA. A p-value <0.05 was considered significant.

Quantification of TNF-α release with wear debris particles: Macrophages were cultured as above. The media was then replaced with 1 ml of media containing one of three conditions: 1) media alone; 2) PMMA particles at a dose of 0.30% v/v; 3) 0.30% PMMA particles plus 10 μg/ml of polymyxin B. TNF-α levels were measured at 1, 4, and 12 hours post challenge.

### Results

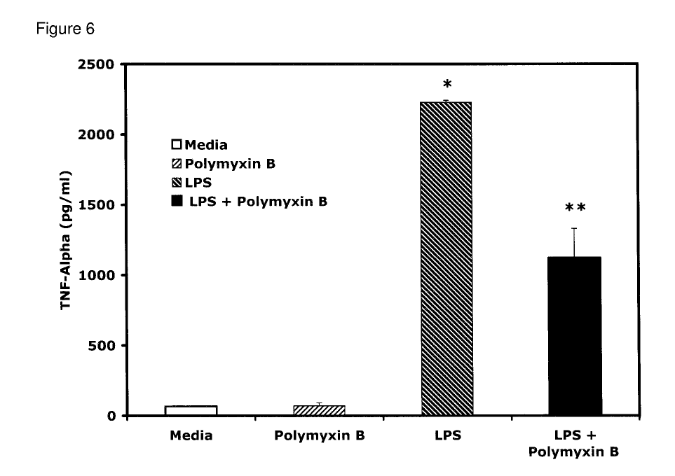

Macrophages incubated with polymyxin B for 12 h had no significant difference in TNF-α release compared to macrophages alone (FIG. 6). The addition of LPS markedly increased TNF-α release. Polymyxin B markedly decreased the effects of LPS. LPS alone resulted in TNF-α levels above the upper range of the assay (2250 pg/ml). The decrease with polymyxin B to 1120 pg/ml therefore represents at least a 50% reduction in TNF-α release.

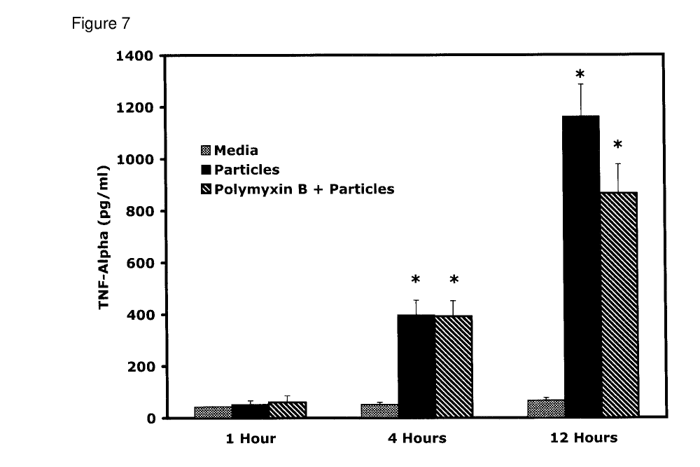

Macrophages incubated without particles or LPS had very low levels of TNF-α release. Macrophages incubated with PMMA particles alone had marked time-dependent increases in TNF-α release (FIG. 7). The addition of polymyxin B to particles did not alter TNF-α release at 4 h and produced a non-significant 25% decrease at 12 h.

This study using the Raw264.7 macrophage cell line demonstrated that the majority of the inflammatory response to PMMA particles is not simply attributable to endotoxin contamination but rather is due to the particles themselves. By neutralizing endotoxin, polymyxin B decreased LPS-induced TNF-α release in the absence of particles by at least 50%. In contrast, polymxyin B had no significant effect on particle-induced TNF-α release. The washing protocol for the PMMA particles used in this study appears to effectively remove any residual adsorbed endotoxin without altering the ability of the particles to activate macrophages. The exquisite sensitivity of macrophages to LPS (marked activation at LPS concentration of 500 pg/ml) is consistent with studies which suggest that LPS contamination may be responsible for macrophage activation in some particle experiments. However, by neutralizing LPS without needing harsh treatments to remove LPS, our results demonstrate that particles alone, without residual LPS, are sufficient to produce intense macrophage activation.

The disclosures of Pearl et al. (2008) Transactions of the 54th Annual Meeting of the Orthopaedic Research Society 750, “Role of The MyD88 pathway in activation of macrophages By PMMA particles”; Pearl et al. (2008) 8th World Biomaterials Congress, Amsterdam, Netherlands, “Role of the MyD88 pathway in particle-induced macrophage activation”; and Pearl et al. (2008) 8th World Biomaterials Congress, Amsterdam, Netherlands, “Macrophages can recognize characteristics of PMMA particles independent of adsorbed endotoxins” are herein specifically incorporated by reference.

What is claimed is:

1. A method for treating a particle induced inflammatory disease in a patient, the method comprising:
administering to said patient a therapeutically effective dose of an agent that inhibits MyD88 activity, wherein the severity of the disease is decreased.

2. The method according to claim 1, wherein said disease is inflammation associated with orthopedic biomaterial wear debris.

3. The method according to claim 1, wherein the disease is sepsis.

4. The method according to claim 1, wherein said agent is a peptide or peptidomimetic that inhibits MyD88 dimerization.

5. The method of claim 2, wherein the treatment is initiated following implantation of an orthopedic device.
